(19) United States
(12) Patent Application Publication
(10) Pub. No.: US 2024/0115388 A1
(43) Pub. Date: Apr. 11, 2024

(54) DEVICES AND METHODS FOR DETERMINING CLIP SIZE FOR LEFT ATRIAL APPENDAGE OCCLUSION

(71) Applicant: AtriCure, Inc., Mason, OH (US)

(72) Inventors: Frank FAGO, Mason, OH (US); Ed BIEHLE, West Chester, OH (US); David HOLTMEIER, Villa Hills, KY (US)

(73) Assignee: AtriCure, Inc., Mason, OH (US)

(21) Appl. No.: 18/484,230

(22) Filed: Oct. 10, 2023

Related U.S. Application Data
(60) Provisional application No. 63/378,948, filed on Oct. 10, 2022.

(51) Int. Cl. A61F 2/24

(52) U.S. Cl. CPC A61F 2/2496; A61F 2240/001; A61F 2250/0097

(57) ABSTRACT

Disclosed herein are devices and methods for a reusable clip sizing tool to select a clip for use with a left atrial appendage. The clip can comprise a handle portion having a proximal end and a distal end, a base portion distal to the handle portion, a plurality of indicator markers on the base portion, wherein the plurality of indicator markers comprise numbers corresponding to a size of a left atrial appendage when a distal end of the base portion is aligned with the left atrial appendage. The plurality of indicator markers can comprise alternating contrast levels. The handle portion and the base portion can comprise a malleable material.

### CROSS REFERENCE TO RELATED APPLICATION

This application claims the benefit of U.S. Provisional Patent Application No. 63/378,948 filed on Oct. 10, 2022.

### FIELD OF TECHNOLOGY

The present disclosure relates generally to the field of applying clips, and more specifically, selecting an appropriately sized clip for the appendage morphology.

### BACKGROUND

Left atrial appendage closure procedures often use a clip for closing the appendage at its base. Prior to the procedure, a sizing tool can be used to determine the size of the clip to be applied. Currently, single-use clip sizing tools are supplied with clip appliers used for such procedures. However, single use tools can be expensive, with the added cost of packaging and sterilization. Further, not all users elect to use a pre-packaged tool, which can lead to economic and material waste.

In addition, the sizing tool contacts the patient and becomes a biohazard. Accordingly, recycling the tool may not be permissible in certain situations. Additionally, the packaging contributes to the medical waste burden.

Therefore, there remains a need for methods and devices for reusable sizing tools that can be autoclaved at the hospital per a reusable device sterilization program.

### SUMMARY

Disclosed herein are devices and methods for a reusable clip sizing tool to select a clip for use with a left atrial appendage. The clip can comprise a handle portion having a proximal end and a distal end, a base portion distal to the handle portion, a plurality of indicator markers on the base portion, wherein the plurality of indicator markers comprise numbers corresponding to a size of a left atrial appendage when a distal end of the base portion is aligned with the left atrial appendage. The plurality of indicator markers can comprise alternating contrast levels. The handle portion and the base portion can comprise a malleable material.

The tool can further comprise an intermediate portion between the handle portion and the base portion, wherein the intermediate portion comprises a bend. The numbers can be aligned in a portrait direction from the distal end of the base portion to a proximal end of the base portion such that a bottom of the numbers face proximally. The malleable material can comprise annealed stainless steel configured to resist deterioration from autoclave and chemical sterilization and to maintain bendability after use. The distal end of the base portion can be atraumatic. The tool can be made from a flat wire fabrication method. The plurality of indicator markers can be configured to withstand multiple sterilization cycles. The plurality of indicator markers are made by laser engraving, chemical etching, stamping, or pad printing. The plurality of indicator markers can comprise contrasting inverted background patterns between adjacent sizes.

### BRIEF DESCRIPTION OF THE DRAWINGS

FIG. 1A illustrates a perspective view of a sizing tool according to one variation of the invention.

FIG. 1B illustrates a view of the base portion of the sizing tool of FIG. 1A.

FIG. 2 illustrates a close-up view of the sizing tool adjacent to the left atrial appendage of a patient.

FIG. 3 illustrates a perspective view of a sizing tool according to yet another variation of the invention.

FIG. 4 illustrates a perspective view of a sizing tool according to yet another variation of the invention.

FIG. 5 illustrates a perspective view of a sizing tool according to yet another variation of the invention.

FIG. 6 illustrates a perspective view of a sizing tool according to yet another variation of the invention.

FIG. 7 illustrates a perspective view of a sizing tool according to yet another variation of the invention.

FIG. 8 illustrates a diagram of a viewing comparison between tools of varying thicknesses.

FIG. 9 illustrates a flat wire fabrication method for manufacturing the sizing tool.

### DETAILED DESCRIPTION

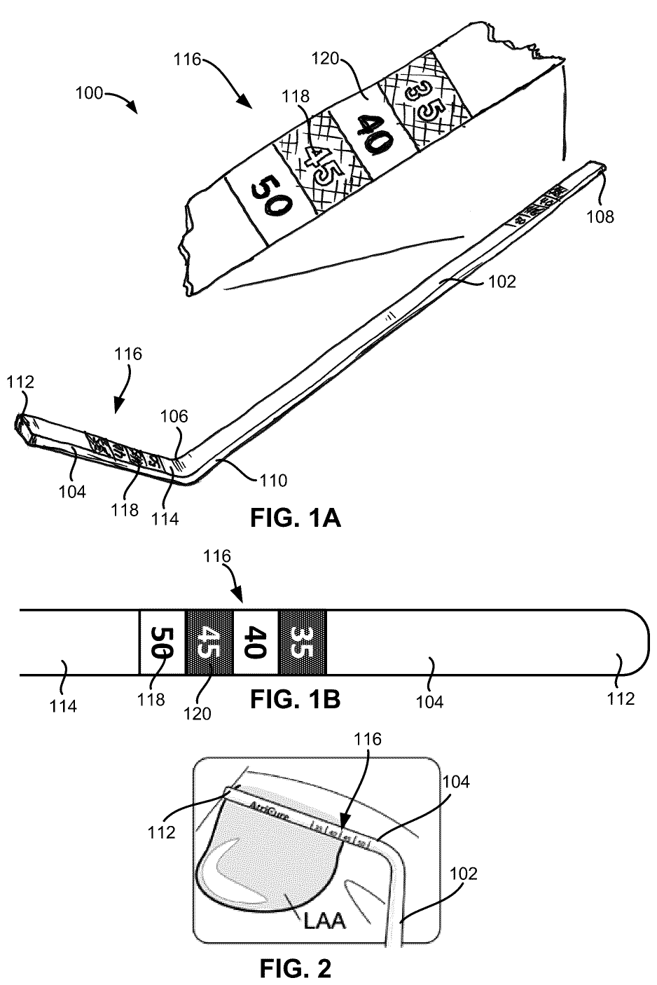

FIG. 1A illustrates a perspective view of a sizing tool 100 according to one variation of the invention. The tool 100 comprises a handle portion 102, a base portion 104, and an intermediate portion 106 between the handle portion 102 and the base portion 104. The handle portion 102 has a handle portion proximal end 108 for handling by a user and a handle portion distal end 110 at which the intermediate portion 106 is located. The base portion 104 has a base portion distal end 112 and a base portion proximal end 114 at which the intermediate portion 106 is located.

For minimally invasive thoracic procedures, the tool 100 is configured to fit through a 5 mm trocar. The tool 100 can have a height between about 0.03 inches and about 0.09 inches (e.g., about 0.06 inches) and a width between about 0.15 inches and about 0.2 inches (e.g., about 0.1875 inches). The handle portion 102 can have a length between about 10 inches to about 15 inches (e.g., 11.5 inches). The base portion 104 can have a length between about 2 inches to about 3 inches (e.g., 2.5 inches).

FIG. 1B illustrates a view of the base portion 104 of the sizing tool 100 of FIG. 1A. The base portion 104 comprises one or more indicator markers 116 for determining the size of a clip to be applied to a left atrial appendage. The indicator markers 116 can comprise sequential numbers 118 (e.g., 35, 40, 45, 50) which represent a clip size in millimeters. Bottoms of the numbers 118 face proximally towards the intermediate portion 106 such that the user can easily read the numbers during use (i.e., the numbers are aligned in a portrait direction). To further enhance visibility for the user and to compensate for glare from a light source of an endoscope, adjacent numbers 118 also have levels of inverted and/or contrasting backgrounds 120 (e.g., light and dark), as also seen in the close-up view of FIG. 1A. Alternatively or in addition, the backgrounds 120 can comprise different shading patterns or cross-hatching patterns. Further, the numbers 118 can alternate in size (i.e., font size) for improved visualization.

The backgrounds 120 and numbers 118 can be made via pad printing, 3D printing, metal injection molding, stamping (coining), laser marking, chemical etching, or any combinations thereof. Such methods can also be used to add important information onto tool 100 (e.g., reference codes, part number codes, company logos). Alternatively, the base portion 104 or the handle portion 102 can be machined from bar stock on a screw machine with the indicator markers 116 engraved.

The handle portion 102 comprises a greater length than the base portion 104. The handle portion 102 can also comprise indicator markers 116 having numbers 118 for sizing at the handle portion distal end 110. This allows for the user to size various anatomical regions and to compensate for varying anatomy. Accordingly, the handle portion and the base portion 104 can be atraumatic.

The tool 100 is made of a malleable material (e.g., stainless steel) and/or ductile material to allow the tool 100 bendability. The user can bend the tool at any malleable section to achieve various viewing angles and measurement angles as necessary. The tool 100 can alternatively be made of annealed aluminum or any suitable plastic.

The tool 100 is reusable due to its annealed, 300-series stainless steel material (e.g., 303 stainless steel). The material is made to resist chemicals and damage or deterioration from autoclave techniques for sterilization. Accordingly, the tool 100 can be autoclaved within steam under pressure to kill any living bacteria, viruses, fungi, and spores, during which the tool 100 is heated to an elevated sterilization temperature for a pre-determined amount of time. As such, the tool 100 maintains its bendability after withstanding multiple sterilization cycles. The yield strength of the material can be about 35,000 psi.

FIG. 2 illustrates a close-up view of the sizing tool 100 adjacent to the left atrial appendage (LAA) of a patient. During use, the user can guide the tool 100 such that the base portion distal end 112 is aligned and adjacent to the LAA. Once the tool 100 is in position, the user views (i.e., via an endoscope) the appropriate clip size for the specific LAA.

It should be noted that the actual clip size range can be at or lower than the number 118 on the base portion 104 to account for irregularities in the appendage or for appendages that are already clipped during the measurement, increasing their length. The LAA is naturally oval in a free state, so when a clip forces it closed, it inherently gets longer. Additionally, the tissue in the appendage squishes under the pressure of the clip which further adds to the length. The sizing tool 100 compensates for the squish by allowing a measurement factor of about 3 mm less than the actual size of the clip.

The user can also use the tool 100 for manipulation of the LAA or surrounding anatomy for evaluation. The bendability of the tool 100 improves the ability for the user to manipulate the anatomy as needed for accurate measurement.

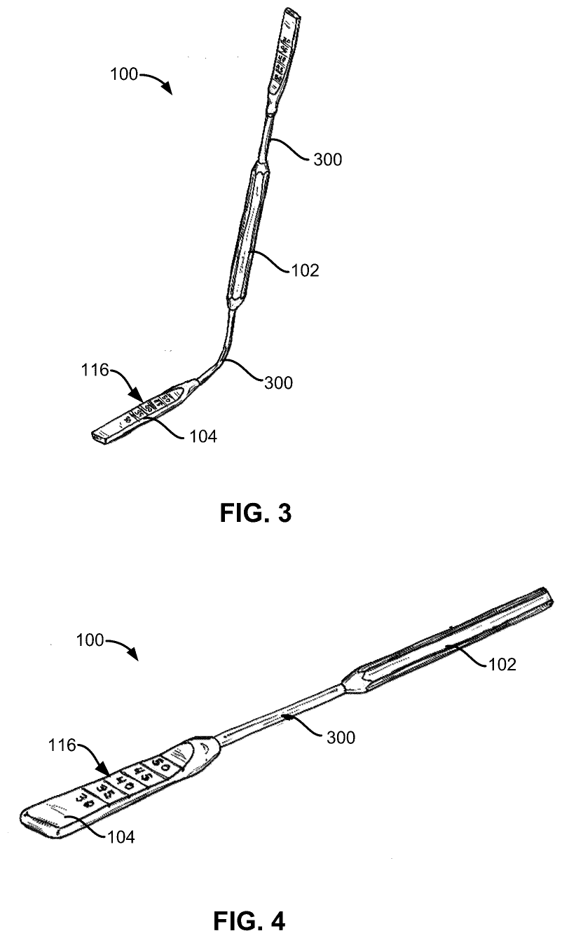

FIG. 3 illustrates a perspective view of a sizing tool 100 according to yet another variation of the invention. The tool 100 can comprise a handle portion 102 surrounded by malleable portions 300. The malleable portions 300 offer increased bendability at their respective locations along the tool 100. The malleable portions 300 can have a circular cross-section and can be thinner than the handle portion 102. The malleable portions 300 can comprise 300 series annealed stainless steel and can have a diameter of about 0.125 inches. The malleable portions 300 provide fewer or no creases that can be difficult to clean.

The base portion 104 and a handle portion proximal end 108 can comprise indicator markers 116 having numbers 118 and at which the tool 100 is shaped like a paddle to aid in navigation through the anatomy. The handle portion 102 comprises a hexagonal grip which provides a tactile feel for the user especially in wet operating fields as well as facilitated cleaning of the tool 100. Alternatively, the handle portion 102 can comprise a knurled configuration for gripping and for added friction in a wet environment. The hexagonal grip allows the tool 100 to be free of crevices where organic material can be trapped and be difficult to clean.

FIG. 4 illustrates a perspective view of a sizing tool 100 according to yet another variation of the invention. In this variation, the tool 100 comprises a handle portion 102 with a hexagonal grip, a base portion 104 with indicator markers 116, and a malleable portion therebetween. The malleable portion 300 can have a circular cross-section and can be thinner than the handle portion 102. The tool 100 in this variation can be substantially straight in its default configuration and accordingly, more rigid throughout its length. The tool 100 can be used in conjunction with a clip applier and can be sized accordingly.

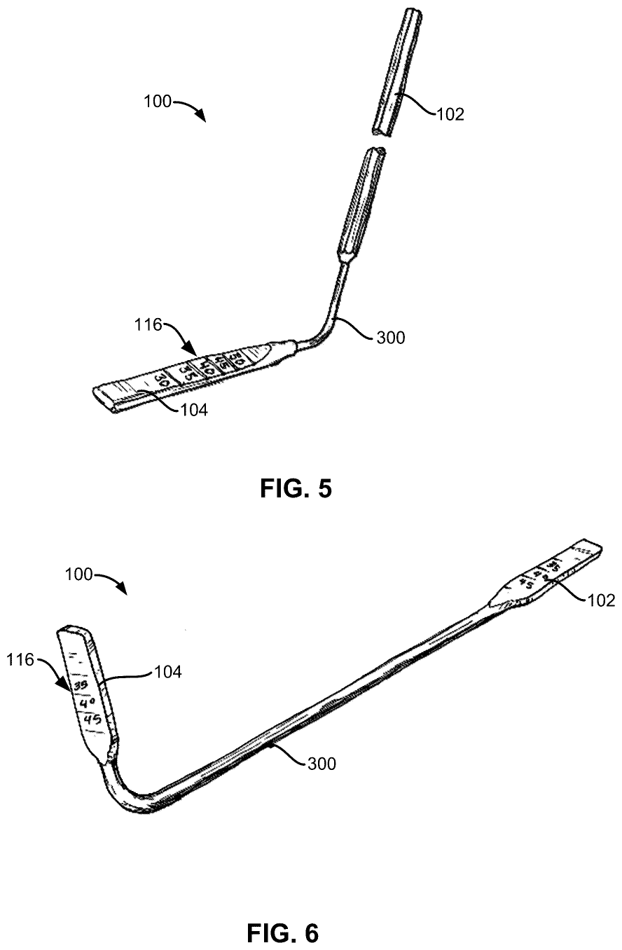

FIG. 5 illustrates a perspective view of a sizing tool 100 according to yet another variation of the invention. In this variation, the tool 100 comprises a handle portion 102 with a hexagonal grip, a base portion 104 with indicator markers 116, and a malleable portion therebetween. The malleable portion 300 can have a circular cross-section and can be thinner than the handle portion 102. The tool 100 in this variation can be substantially bent at an angle in its default configuration (e.g., L-shaped).

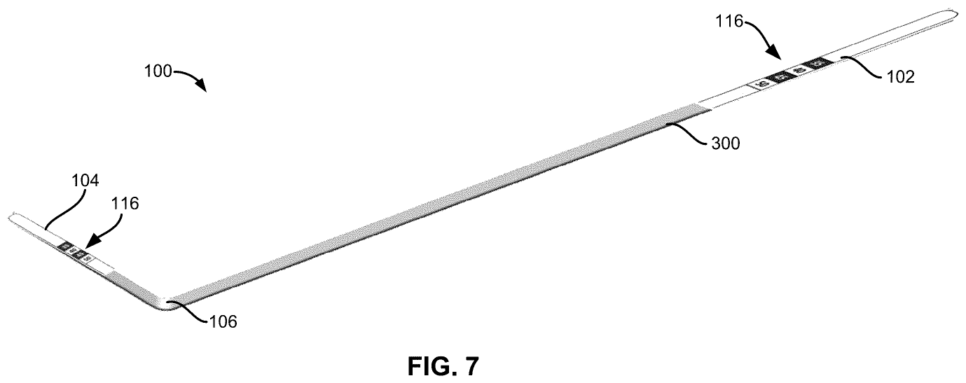

FIG. 6 illustrates a perspective view of a sizing tool 100 according to yet another variation of the invention. In this variation, the tool 100 comprises a handle portion 102 having indicator markers 116 at a handle portion proximal end 108, a base portion 104 with indicator markers 116, and a malleable portion therebetween. The malleable portion 300 can have a circular cross-section. The base portion 104 and a handle portion proximal end 108 can comprise indicator markers 116 having numbers 118 and at which the tool 100 is shaped like a paddle to aid in navigation through the anatomy. The tool 100 in this variation can be substantially bent at an angle in its default configuration. The base portion 104 in this variation can have a width between about 0.1 inches to about 0.3 inches (e.g., about 0.21 inches).

FIG. 7 illustrates a perspective view of a sizing tool 100 according to yet another variation of the invention. In this variation, the tool 100 comprises a handle portion 102 with indicator markers 116, a base portion 104 with indicator markers 116, and a malleable portion 300 therebetween. The tool 100 can be manufactured from a single part and can be substantially flat along its entire length. The tool 100 in this variation can be substantially bent at an angle in its default configuration.

The tool 100 can be laser cut to facilitate bending and can be forged from a single part. Alternatively, the tool 100 can bend via a hinge mechanism at the intermediate section or any other malleable portion 300.

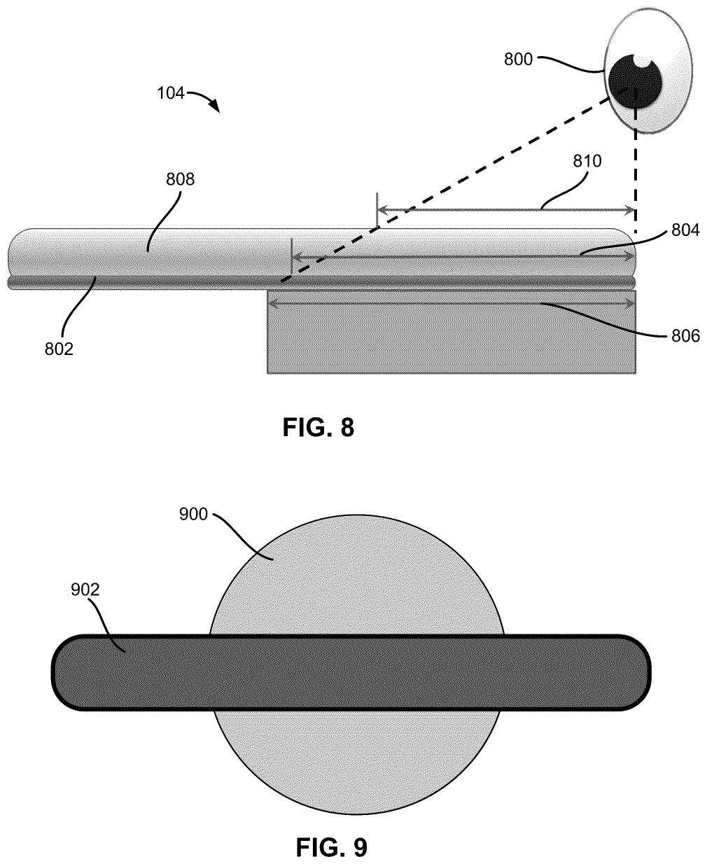

FIG. 8 illustrates a diagram of a viewing comparison between tools of varying thicknesses. A first tool 808 having a larger thickness is shown alongside a second tool 802 having a smaller thickness. The viewer 800 looking through an endoscope will experience parallax, the apparent displacement or the difference in apparent length of an object as seen from two different points not on a straight line with the object. As seen in FIG. 8, the apparent length 804 of the thinner tool 802 is closer to the actual length 806 of the thinner tool 802 than the apparent length 810 of the thicker tool 808. This minimizes parallax, which is helpful for uses with an endoscope which utilizes a wide-angle lens to improve the field of view. Accordingly, the user can view the thinner tool 802 without fear of a high degree of inaccuracy due to parallax.

FIG. 9 illustrates a flat wire fabrication method for manufacturing the sizing tool 100. The tool 100 can be produced from flat wire to eliminate concern for sharp edges longitudinally. Flat wire is produced by flattening round wire stock, such that the edges are radiused. Flattening the round wire stock hardens the material such that it is ready to be subsequently annealed. Accordingly, the tool 100 is configured to be as atraumatic as possible to minimize inadvertent injury.

The starting wire 900 can have a diameter between about 0.1 inches and about 0.15 inches (e.g., about 0.116 inches). The flattened wire 902 can have a height between about 0.03 inches and about 0.09 inches (e.g., about 0.06 inches). The flattened wire 902 can have a width between about 0.15 inches and about 0.2 inches (e.g., about 0.188 inches). The flattened wire 902 can have an area between about 0.01 squared inches and about 0.02 squared inches (e.g., about 0.105 squared inches).

The flattened wire 902 can be drawn into a flat shape and cut to length during manufacturing. Next, a cutoff die forms the radii via punch/die removal of the sharp edges, creating blunt edges at the ends of the tool 100. Any excess material left over from the cutoff die can be bead blasted away resulting in blunt rounded edges. Finishing can also be accomplished by a machining operation, swaging, hand deburring, tumble deburring, or electro-polishing.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

It will be understood by one of ordinary skill in the art that the various methods disclosed herein may be embodied in a non-transitory readable medium, machine-readable medium, and/or a machine accessible medium comprising instructions compatible, readable, and/or executable by a processor or server processor of a machine, device, or computing device. The structures and modules in the figures may be shown as distinct and communicating with only a few specific structures and not others. The structures may be merged with each other, may perform overlapping functions, and may communicate with other structures not shown to be connected in the figures. Accordingly, the specification and/or drawings may be regarded in an illustrative rather than a restrictive sense.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

We claim:

1. A reusable clip sizing tool, the tool comprising:
a handle portion having a proximal end and a distal end;
a base portion distal to the handle portion;
a plurality of indicator markers on the base portion, wherein the plurality of indicator markers comprise numbers corresponding to a size of a left atrial appendage when a distal end of the base portion is aligned with the left atrial appendage;
wherein the plurality of indicator markers comprise alternating contrast levels;
wherein the handle portion and the base portion comprise a malleable material.

2. The tool of claim 1, further comprising an intermediate portion between the handle portion and the base portion, wherein the intermediate portion comprises a bend.

3. The tool of claim 1, wherein the numbers are aligned in a portrait direction from the distal end of the base portion to a proximal end of the base portion such that a bottom of the numbers face proximally.

4. The tool of claim 1, wherein the malleable material comprises annealed stainless steel.

5. The tool of claim 1, wherein the malleable material is configured to resist deterioration from autoclave and chemical sterilization.

6. The tool of claim 1, wherein the malleable material is configured to maintain bendability after use.

7. The tool of claim 1, wherein the distal end of the base portion is atraumatic.

8. The tool of claim 1, wherein the tool is made from a flat wire fabrication method.

9. The tool of claim 1, wherein the plurality of indicator markers are configured to withstand multiple sterilization cycles.

10. The tool of claim 9, wherein the plurality of indicator markers are made by laser engraving, chemical etching, stamping, or pad printing.

11. The tool of claim 1, wherein the plurality of indicator markers comprise contrasting inverted background patterns between adjacent indicator markers.
